(19) United States
(12) Patent Application Publication
(10) Pub. No.: US 2023/0210812 A1
(43) Pub. Date: Jul. 6, 2023

(54) DIHYDROMYRICETIN NANOPARTICLE FORMULATIONS

(71) Applicants: The Trustees of Princeton University, Princeton, NJ (US); Cheers Health, Inc., Houston, TX (US)

(72) Inventors: Robert K. PRUD'HOMME, Princeton, NJ (US); Brooks POWELL, Houston, TX (US); Chang TIAN, Princeton, NJ (US)

(73) Assignees: The Trustees of Princeton University, Princeton, NJ (US); Cheers Health, Inc., Houston, TX (US)

(21) Appl. No.: 17/899,157

(22) Filed: Aug. 30, 2022

Related U.S. Application Data
(63) Continuation of application No. 16/810,710, filed on Mar. 5, 2020, now abandoned.
Application No. 16/810,710 is a continuation of application No. PCT/US2018/049580, filed on Sep. 5, 2018.
(60) Provisional application No. 62/554,897, filed on Sep. 6, 2017.

(51) Int. Cl. A61K 9/51; A61K 31/352; A61K 33/26; A61K 33/34; A61K 33/06

(52) U.S. Cl. CPC A61K 9/5161; A61K 31/352; A61K 9/5146; A61K 33/26; A61K 33/34; A61K 33/06; B82Y 5/00

(57) ABSTRACT

Compositions that increase the bioavailability of dihydromyricetin are presented. The bioavailability is increased by methods including formulating dihydromyricetin in nanoparticle form, delivering dihydromyricetin with permeabilizers, and encapsulating dihydromyricetin with an enteric coating.

This application is a continuation of U.S. application Ser. No. 16/810,710, filed Mar. 5, 2020, which is a continuation of International Application No. PCT/US2018/049580, filed Sep. 5, 2018, which claims the benefit of U.S. Provisional Application No. 62/554,897, filed Sep. 6, 2017, all of which are hereby incorporated by reference in their entireties herein.

### FIELD OF THE INVENTION

The present invention relates to dihydromyricetin nanoparticles having enhanced bioavailability and a process of making such dihydromyricetin nanoparticles.

### BACKGROUND OF THE INVENTION

Dihydromyricetin (DHM), a flavonoid compound isolated from the Hovenia plant can “sober-up” rats inebriated with alcohol, prevent predisposed rats from becoming alcoholics, return alcoholic rats to baseline levels of alcohol consumption, reduce hangover symptoms (Shen, Y.; Lindemeyer, A. K.; Gonzalez, C.; Shao, X. M.; Spigelman, I.; Olsen, R. W.; Liang, J., “Dihydromyricetin as a novel anti-alcohol intoxication medication”, Journal of Neuroscience 2012, 32 (1), 390-401), and prevent fetal alcohol spectrum disorders in the offspring of rats exposed to significant amounts alcohol during pregnancy (Liang, J.; Shen, Y.; Shao, X. M.; Scott, M. B.; Ly, E.; Wong, S.; Nguyen, A.; Tan, K.; Kwon, B.; Olsen, R. W., “Dihydromyricetin prevents fetal alcohol exposure-induced behavioral and physiological deficits: the roles of GABA_{A} receptors in adolescence”, Neurochemical Research 2014, 39 (6), 1147-1161).

### SUMMARY OF THE INVENTION

In an embodiment of the invention, a nanoparticle includes dihydromyricetin complexed with a metal. The dihydromyricetin can be in an amorphous state. The metal can be iron, e.g., in the iron(III) state or the iron(II) state, copper, e.g., in the copper(II) state, magnesium, e.g., in the magnesium(II) state, or combinations. The nanoparticle can further include an amphiphilic stabilizer, for example, an ethoxylated sugar surfactant, a block copolymer, polyethylene oxide-block-polypropylene oxide (PEO-b-PPO), a zein-casein protein mixture, gelatin, derivatized cellulosic polymer, hydroxypropyl methylcellulose with succinic anhydride substitution, polyethylene glycol (PEG) functionalized vitamin E (tocopherol succinate PEG), polystyrene-block-polyethylene glycol (PS-b-PEG), hydroxypropyl methylcellulose (HPMC), hydroxypropyl methylcellulose acetate succinate (HPMCAS), or combinations of these. For example, the stabilizer can have a molecular weight of from about 1, 2, 2.5, 3, 4, 5, 6, 6.6, 7, 10, 15, 20, 25, 30, 35, 40, 45, 50, 60, 80, 100, 150, or 200 kDa to about 2, 2.5, 3, 4, 5, 6, 6.6, 7, 10, 15, 20, 25, 30, 35, 40, 45, 50, 60, 80, 100, 150, 200, or 500 kDa. For example, in a block copolymer stabilizer, the less hydrophilic or hydrophobic block can have a molecular weight of from about 0.2, 0.5, 1, 1.2, 1.5, 1.6, 1.8, 2, 2.5, 3, 4, 5, 7, 10, 15, 20, 25, 30, 35, 40, 45, 50, 60, 80, 100, 150, or 200 kDa to about 0.5, 1, 1.2, 1.5, 1.6, 1.8, 2, 2.5, 3, 4, 5, 7, 10, 15, 20, 25, 30, 35, 40, 45, 50, 60, 80, 100, 150, 200, or 250 kDa, and the more hydrophilic or hydrophilic block can have a molecular weight of from about 0.2, 0.5, 1, 1.2, 1.5, 1.8, 2, 2.5, 3, 4, 5, 7, 10, 15, 20, 25, 30, 35, 40, 45, 50, 60, 80, 100, 150, or 200 kDa to about 0.5, 1, 1.2, 1.5, 1.8, 2, 2.5, 3, 4, 5, 7, 10, 15, 20, 25, 30, 35, 40, 45, 50, 60, 80, 100, 150, 200, or 250 kDa.

The nanoparticle can further include a cyclodextrin. The nanoparticle can further include a permeabilizer, for example, a fatty acid, a saturated fatty acid, capryic acid, a capryate salt, a fatty acid complexed with a cation, such as a metal cation, magnesium, calcium, or zinc divalent cation, or iron trivalent cation, or combinations of these. The nanoparticle can be encapsulated by an enteric coating, such as a polymeric coating, for example, a methacrylate copolymer coating. For example, the nanoparticle can have a diameter of at least 5, 10, 20, 25, 50, 60, 100, 110, 120, 150, 170, 180, 200, 210, 250, 300, 400, 500, 1000, or 2000 nm and at most 10, 20, 25, 50, 60, 100, 110, 120, 150, 170, 180, 200, 210, 250, 300, 400, 500, 1000, 2000, or 5000 nm. For example, the nanoparticle can have a diameter in the range of from 100 nm to 5000 nm. For example, the nanoparticle can have a diameter in the range of from 500 nm to 1000 nm. For example, the nanoparticle can have a diameter in the range of from 10 nm to 1000 nm, in the range of from 20 nm to 500 nm, in the range of from 25 nm to 400 nm, in the range of from 60 nm to 400 nm, or in the range of from 100 to 250 nm.

In an embodiment of the invention, the nanoparticle is included in an oral dosage form. The oral dosage form can be formed of a multitude of nanoparticles. The oral dosage form can include a permeabilizer. The oral dosage form can be encapsulated by an enteric coating.

In a method according to the invention, a dihydromyricetin nanoparticle is formed by dissolving dihydromyricetin in an organic solvent to form an organic solution and continuously mixing the organic solution with an aqueous stream to form a mixed solution from which the dihydromyricetin nanoparticle assembles and precipitates; Flash NanoPrecipitation can be used to form the dihydromyricetin nanoparticle. The aqueous stream can include a metal cation. The aqueous stream can include a metal halide, such as an iron halide. The aqueous stream can include an iron (Fe) salt, such as iron(III) chloride (Fe(III)Cl₃) and/or iron(II) chloride (Fe(II)Cl₂), a copper (Cu) salt, such as copper(II) chloride (Cu(II)Cl₂), or a magnesium (Mg) salt, such as magnesium(II) chloride (Mg(II)Cl₂), or combinations. The mixed solution can be collected in a reservoir that optionally contains a buffer, such as phosphate-buffered saline (PBS) buffer, and/or a base, such as ammonia or an ammonium base. The aqueous stream can include an amphiphilic stabilizer and/or a polymeric stabilizer. The polymeric stabilizer can be a polystyrene-block-polyethylene glycol (PS-b-PEG). The amphiphilic stabilizer can include an ethoxylated sugar surfactant, a block copolymer, polyethylene oxide-block-polypropylene oxide (PEO-b-PPO), a zein-casein protein mixture, gelatin, derivatized cellulosic polymer, hydroxypropyl methylcellulose, hydroxypropyl methylcellulose with succinic anhydride substitution, polyethylene glycol (PEG) functionalized vitamin E (tocopherol succinate PEG), or combinations of these. The organic solution can include a permeabilizer, for example, capryic acid, capryate salts, or combinations of these. The organic solution can include a material that forms an enteric coating. In the method, a plurality (multitude) of dihydromyricetin nanoparticles can be formed; the nanoparticles can be aggregated with an enteric coating. The organic solvent can include methanol, ethanol, n-propanol, isopropanol, acetone, ethyl acetate, tetrahydrofuran (THF), or combinations of these. The organic solution can include an organic base, for example, pyridine. The aqueous stream can include a base, for example, ammonia, an ammonium compound, and/or a hydroxide base, such as sodium hydroxide and/or potassium hydroxide. The organic solution can include an amphiphilic stabilizer and/or a polymeric stabilizer. The polymeric stabilizer can be a polystyrene-block-polyethylene glycol (PS-b-PEG). The amphiphilic stabilizer can include an ethoxylated sugar surfactant, a block copolymer, polyethylene oxide-block-polypropylene oxide (PEO-b-PPO), a zein-casein protein mixture, gelatin, derivatized cellulosic polymer, hydroxypropyl methylcellulose, hydroxypropyl methylcellulose with succinic anhydride substitution, polyethylene glycol (PEG) functionalized vitamin E (tocopherol succinate PEG), or combinations of these. A cyclodextrin can be added to the dihydromyricetin nanoparticle to form a mixture and the cyclodextrin-dihydromyricetin nanoparticle mixture can be lyophilized.

A dihydromyricetin nanoparticle can be spray dried to yield a dry powder. A sugar, trehalose, maltodextrin, sucrose, mannitol, leucine, casein, a starch or a cellulosic polymer can be added prior to spray drying.

A dihydromyricetin nanoparticle can be prepared by dissolving dihydromyricetin in an organic solvent to form an organic solution, and by continuously mixing the organic solution with an aqueous stream to form a mixed solution from which the dihydromyricetin nanoparticle assembles and precipitates.

A nanoparticle or dihydromyricetin nanoparticle according to the invention can be used as a medicament. For example, such a nanoparticle or dihydromyricetin nanoparticle can be used to reduce hangover symptoms, prevent an alcohol use disorder, prevent alcoholism, treat an alcohol use disorder, treat alcoholism, and/or treat an alcohol overdose. For example, such a nanoparticle or dihydromyricetin nanoparticle can be used to increase antioxidant capacity. For example, such a nanoparticle or dihydromyricetin nanoparticle can be used to used in neuroprotection, for example, in the context of Alzheimer's and/or Parkinson's diseases. For example, such a nanoparticle or dihydromyricetin nanoparticle can be used to inhibit inflammation. For example, such a nanoparticle or dihydromyricetin nanoparticle can be used to protect the kidney, the liver, and/or another organ. For example, such a nanoparticle or dihydromyricetin nanoparticle can be used to prevent a cancer or treat a cancer. For example, such a nanoparticle or dihydromyricetin nanoparticle can be used to prevent, ameliorate, or treat a metabolic disorder, such as diabetes, weight gain, hyperlipidemia, and/or atherosclerosis. For example, such a nanoparticle or dihydromyricetin nanoparticle can be used to treat a bacterial infection, for its anti-bacterial activity, and/or as an antibiotic.

### DESCRIPTION OF THE FIGURES

FIG. 1A shows the size distribution of DHM nanoparticles (NPs) made using a concentration of iron(III) chloride (FeCl₃) in the aqueous (water) stream of 1 mg/mL and concentrations of DHM in the organic (THF) stream of from 1 mg/mL to 10 mg/mL.

FIG. 1B shows the size distribution of DHM nanoparticles (NPs) made using a concentration of iron(III) chloride (FeCl₃) in the aqueous (water) stream of 2 mg/mL and concentrations of DHM in the organic (THF) stream of from 1 mg/mL to 8 mg/mL.

FIG. 2 shows the size distribution of DHM nanoparticles made using different metal ions. NPs made using Fe(III), Fe(II), Cu(II), and Mg(II) have z-average sizes of 110, 150, 60, and 25 nm, respectively.

FIG. 3 shows the ultraviolet-visible (UV-Vis) absorbance spectrum for a solution of DHM nanoparticles formed using iron(III) (Fe(III)). The absorbance at 295 nm was 0.8444. The absorbance at 325 nm was 0.7231. Let the concentration of free unbound DHM be C1 and unencapsulated DHM bound to iron(III) be C2. Then form a system of simultaneous equations using the absorbance over DHM concentration at 295 and 325 nm. C1=0.0076 mg/mL, and C2=0.0161 mg/mL. Given that the total DHM concentration coming out of the CU was 0.1 mg/mL, the encapsulation efficiency for this formulation was 76%.

FIG. 4 shows that the absorbance spectrum of DHM was shifted by changing the pH of its solution. DHM solution without pH adjustment has a pH of 5.8, with two peaks appearing at 290 nm and 325 nm. By adding acid or base, one of the two peaks is eliminated, so that the concentration of DHM is only related to the absorbance of one wavelength.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are hereby incorporated by reference in their entirety as if each had been individually incorporated.

DHM demonstrates the pharmacological properties expected to underlie successful medical treatment of alcohol use disorders (AUDs) (Shen, Y. et al. 2012; Liang, J. et al. 2014; Davies, D. L.; Bortolato, M.; Finn, D. A.; Ramaker, M. J.; Barak, S.; Ron, D.; Liang, J.; Olsen, R. W., “Recent advances in the discovery and preclinical testing of novel compounds for the prevention and/or treatment of alcohol use disorders”, Alcoholism: Clinical and Experimental Research 2013, 37 (1), 8-15). Given limited available pharmacotherapies for AUDs, of which are also further limited by low patient compliance due to the adverse effects they cause, the advancement of DHM therapeutic strategies is strongly desired (Ji, Y.; Li, J.; Yang, P., “Effects of fruits of Hovenia dulcis Thunb on acute alcohol toxicity in mice”, Zhong yao cai=Zhongyaocai=Journal of Chinese Medicinal Materials 2001, 24 (2), 126-128).

In addition to its potential for AUDs, achieved through inhibiting the effect of alcohol on GABA_{A} receptors (GABA_{A}Rs) in the brain, DHM and the Hovenia plant it is isolated from have shown efficacy in mitigating liver injuries (Fang, H.-L.; Lin, H.-Y.; Chan, M.-C.; Lin, W.-L.; Lin, W.-C., “Treatment of chronic liver injuries in mice by oral administration of ethanolic extract of the fruit of Hovenia dulcis”, The American Journal of Chinese Medicine 2007, 35 (04), 693-703; Hase, K.; Ohsugi, M.; Xiong, Q.; Basnet, P.; Kadota, S.; Namba, T., “Hepatoprotective Effect of Hovenia dulcis Thunb. on Experimental Liver Injuries Induced by Carbon Tetrachloride or D-Galactosamine: Lipopolysaccharide”, Biological and Pharmaceutical Bulletin 1997, 20 (4), 381-385; Ji, Y.; Chen, S.; Zhang, K.; Wang, W., “Effects of Hovenia dulcis Thunb on blood sugar and hepatic glycogen in diabetic mice”, Zhong yao cai=Zhongyaocai=Journal of Chinese Medicinal Materials 2002, 25 (3), 190-191), decreasing alcohol and acetaldehyde concentrations in the blood via enhancing ADH and ALDH activity (Okuma, Y.; Ishikawa, H.; Ito, Y.; Hayashi, Y.; Endo, A.; Watanabe, T., “Effect of extracts from Hovenia dulcis Thunb. alcohol concentration in rats and men administered alcohol”, Journal of Japanese Society of Nutrition and Food Science (Japan) 1995; Wang, X.-Y.; Jiang, Z.-T., RESEARCH PROGRESS IN NATURAL ANTIOXIDANT DIHYDROMYRICETIN [J]. Food Research and Development 2007, 2, 56), and eliminating alcohol-induced excessive free radicals (Okuma, Y. et al. 1995). DHM has been observed to have oxidative stress-mediating activity, i.e., increase antioxidant capacity for scavenging reactive oxygen species, which may result in neuroprotective, nephroprotective (kidney protecting), and hepatoprotective (liver protecting) effects, which may ameliorate, for example, the effects of hypobaric hypoxia, side effects of the chemotherapeutic agent cisplatin, and detrimental effects of ethanol. DHM may have a neuroprotective role in Alzheimer's and Parkinson's diseases. DHM can also inhibit inflammation. DHM can also have anticancer activity and regulate cell proliferation and apoptosis. DHM can mediate metabolism, and may be useful in ameliorating certain metabolic disorders, such as diabetes, weight gain, hyperlipidemia, and atherosclerosis. DHM exhibits anti-bacterial activity (Li, H. et al., “The Versatile Effects of Dihydromyricetin in Health”, Evidence-Based Complementary & Alternative Medicine 2017, Art. ID 1053617).

A DHM formulation designed to reduce many of alcohol's negative effects when taken after alcohol consumption is covered under U.S. Pat. No. 9,603,830 B2 (granted on Mar. 28, 2017) and is sold in the US under the brand name Thrive+®.

Despite promising results in rats, one challenge in translating DHM's efficacy to humans in a commercially viable way is that it has an oral bioavailability of less than 5% (Liu, B.; Du, J.; Zeng, J.; Chen, C.; Niu, S., “Characterization and antioxidant activity of dihydromyricetin—lecithin complex”, European Food Research and Technology 2009, 230 (2), 325-331). DHM is a BCS class IV drug limited by having the properties of both low solubility and permeability. And in the context of successfully commercialized drugs, DHM requires relatively large doses for efficacy. Because DHM is a naturally occurring organic compound isolated from an herb, a nanoencapsulated DHM can be classified as a food (or dietary supplement) under the Dietary products designation using nanoencapsulated DHM as well (Liu, B., 2009).

Three exemplary applications of nanoencapsulated DHM are 1) an alcohol-related health supplement taken before, during, or after alcohol consumption to reduce alcohol's negative health effects and hangovers (similar in concept to applying sunscreen before or during exposure to sun-rays to prevent skin damage and sunburns), 2) an anti-alcoholism drug, and 3) an alcohol-overdose antidote (similar in type to Narcan®, a naloxone-based drug used to prevent death from opioid overdoses).

An alcohol overdose drug could use a quicker route of drug administration, such as injection, nasal spray, or sublingual strip. A new nanoencapsulation formulation for DHM could optimize the route of drug administration. This could produce faster and stronger effects in terms of counteracting an alcohol overdose's life threatening suppression of the central nervous system (CNS) and respiration.

Statistics and reasons demonstrating the need for a supplement and/or drug that could mitigate alcohol's negative effects follow:
Nearly 60% of Americans consume alcohol regularly (at least once a month). These Americans spend on average over $1100 a year on alcohol.
The US CDC (Centers for Disease Control) estimates that the US economy loses $179 billion each year due to lost workplace productivity because of alcohol's negative effects on next-day performance. The CDC estimates that each alcoholic beverage consumed has an average economic opportunity cost of $2.05 (Liu, B., 2009).
The National Council on Alcoholism and Drug Dependency estimates that there are 17.6 million Americans suffering from AUDs.
Depending on how success is defined, the performance of psychosocial methods of reducing alcohol abuse and addiction varies significantly and is not effective for the entire population, thus suggesting the need for an AUD drug to be used in tandem with traditional psychosocial methods for better results.
The US CDC estimates that alcohol's negative effects lead to 88,000 American deaths annually.

### The Target Population:

1) Regular Alcohol Consumers without Need or Desire to Quit:

Nanoencapsulated DHM would serve to reduce the alcohol's economic opportunity cost via reducing losses in next-day performance. Given that Korea's hangover-cure and/or alcohol-related health supplement industry grossed $165 million in 2014 with a population of just 50 million people, we estimate that within a few years US revenues could be well over $500 million. And based on how DHM works to reduce alcohol withdrawal, a major cause of hangovers, DHM also causes less tolerance and dependence to develop as a result of alcohol exposure (Liana, Jing; Olsen, Richard W., “Alcohol use disorders and current pharmacological therapies: the role of GABA_{A} receptors”, Acta Pharmacologica Sinica 2014^{b}, 35 (8), 981-993). From animal studies using DHM, there are strong theoretical grounds for believing that DHM usage after alcohol consumption could reduce one's development of tolerance to and dependence on alcohol. This use is paired with its use for reducing alcohol's other negative health effects, such as alcohol induced liver injury. The economic cost of alcohol-related healthcare is estimated to be $28 billion dollars annually in the US alone (Sacks, Jeffrey J. et al., “National and State Costs of Excessive Alcohol Consumption”, American Journal of Preventive Medicine 2010, 49 (5), e73-e79).

### 2) Alcoholics Desiring to Quit:

Suboxone, similar to methadone in its use as a treatment for opioid addiction, did over $1.4 billion in sales in 2012. There are an estimated 2 million Americans who are addicted to opioids and are in need of treatment. However, there was an estimated 15 million American adults with an alcohol use disorder in 2015—seven times as many as people addicted to opioids.

DHM shares similar characteristics to Suboxone, but for alcohol and the GABA_{A}Rs instead of opioids and opioid receptors. A bioavailable form of DHM based on nanoencapsulation technology that could effectively satiate alcohol withdrawal could be a multi-billion-dollar drug. Used in conjunction with psychosocial-based alcohol rehabilitation treatments and programs, DHM nanoparticles could increase the chances of success.

### 3) Alcohol Overdose Emergencies:

According to the CDC, over 2,000 Americans die each year from alcohol overdoses. By binding to GABA_{A}Rs (GABA_{A} receptors), DHM competes with alcohol and lowers the number of GABA_{A}Rs available for alcohol in the bloodstream to bind to, thereby reducing alcohol's effects on the CNS (central nervous system) (Shen, Y. 2012). Because alcohol is absorbed rapidly into the bloodstream trough the GI tract, medical procedures such as gastric lavage (stomach pumping) are largely ineffective, leaving ventilators as the only viable option in critical situations.

If a fast acting drug delivery method able to deliver an effective dose size of DHM could be developed using nanoencapsulation technology, DHM nanoparticles could be like Naloxone (e.g., Narcan®) but for alcohol. Naloxone-based products sales were over $80 million in 2015. A similar treatment for alcohol overdoses could show similar societal benefits and revenues.

In the market segment of alcohol-related health/hangover CPG products there are several small but growing DHM-based competitors. All current DHM-based products suffer from the same low oral bioavailability.

For AUDs, the current pharmacological methods include the use of benzodiazepine drugs such as diazepam (Valium) to taper someone off the use of GABA_{A} receptor agonists (i.e., alcohol). The use of another addictive drug, such as diazepam, in the treatment of AUDs poses risks. Other AUD drugs have poor patient compliance due to unwanted side effects.

Mechanical ventilation is the only medical procedure than can currently help in life-threatening alcohol overdoses.

As a fast acting drug delivery method able to deliver an effective dose size of DHM, DHM nanoparticles can be like Naloxone (e.g., Narcan®), but for alcohol. Naloxone-based products sales were over $80 million in 2015. A similar treatment for alcohol overdoses could show similar societal benefits and revenues.

In the market segment of alcohol-related health/hangover CPG products there are several small but growing DHM-based competitors. All current DHM-based products suffer from the same low oral bioavailability.

For AUDs, the current pharmacological methods include the use of benzodiazepine drugs such as Valium to taper someone off the use of GABA_{A} receptor agonists (i.e., alcohol). The use of another addictive drug in the treatment of AUDs poses risks. Other AUD drugs have poor patient compliance due to unwanted side effects.

Mechanical ventilation is the only medical procedure than can currently help in life-threatening alcohol overdoses.

### Definitions

A nanoparticle can be defined as a solid core particle with a diameter between 10-5000 nm. The size for particles between 10-600 nm can be measured by dynamic light scattering. The particles analyzed in this patent application are measured using dynamic light scattering in a Malvern Nanosizer, where the size is the z-weighted size reported using the normal mode analysis program provided by the instrument, and the polydispersity index (PDI) is reported based on cumulant analysis of the correlation function. PDI ranges from 0 to 1. For example, the nanoparticles according to the invention can have a PDI of from about 0.02, 0.05, 0.1, 0.11, 0.12, 0.13, 0.14, 0.15, 0.16, 0.17, 0.18, 0.2, 0.21, 0.25, 0.27, 0.3, 0.4, 0.5, 0.6, or 0.8 to about 0.05, 0.1, 0.11, 0.12, 0.13, 0.14, 0.15, 0.16, 0.17, 0.18, 0.2, 0.21, 0.25, 0.27, 0.3, 0.4, 0.5, 0.6, 0.8, or 1.

For mode sizes between 600 and 5000 nm the size can be determined by transmission electron microscopy and can be obtained by measuring on the order of 100 particles and producing a histogram.

A permeabilizer is an agent that enhances the permeation of a drug compound through the epithelial cell layer in the GI tract and, hence, enhances the amount of drug entering the bloodstream.

An enteric coating or enteric polymer is a polymer coating which has a pH dependent solubility. Thereby, the drug can be released at various times and positions in the GI (gastrointestinal) tract, because there are known pH changes in passing through the stomach, small intestines, large intestines, and colon.

Flash NanoPrecipitation is a process that combines rapid micromixing in a confined geometry of miscible solvent and antisolvent streams to effect high supersaturation of components. The resulting high supersaturation results in rapid precipitation and growth of the resulting nanoparticles. A stabilizing agent in the formulation accumulates on the surface of the nanoparticle and halts growth at a desired size. The process has been described in detail in “Process and apparatuses for preparing nanoparticle compositions with amphiphilic copolymers and their use”, B K Johnson, R K Prud'homme, U.S. Pat. No. 8,137,699, granted Mar. 20, 2012. It has further been described in the review article by Saad and Prud'homme (D'addio, S. M.; Prud'homme, R. K., “Controlling drug nanoparticle formation by rapid precipitation”, Advanced Drug Delivery Reviews 2011, 63 (6), 417-426; Saad, W. S.; Prud'homme, R. K., “Principles of nanoparticle formation by flash nanoprecipitation”, Nano Today 2016, 11 (2), 212-227). These references are included in this application in their entirety.

### Technical Approach

Dihydromyricetin (DHM) has low aqueous solubility (0.2 mg/mL at 25° C.), and low permeability through intestinal mucosa (Solanki, S. S.; Sarkar, B.; Dhanwani, R. K., “Microemulsion drug delivery system: for bioavailability enhancement of ampelopsin”, ISRN Pharmaceutics 2012, 2012). It is listed as a biopharmaceutics classification system (BCS) IV drug (Wang, C.; Tong, Q.; Hou, X.; Hu, S.; Fang, J.; Sun, C. C., “Enhancing bioavailability of dihydromyricetin through inhibiting precipitation of soluble cocrystals by a crystallization inhibitor”, Crystal Growth & Design 2016, 16 (9), 5030-5039). Consequently, larger doses of DHM must be administered than if DHM were more readily absorbed by the body. It would be beneficial to increase bioavailability, so that administered doses could be minimized. To that end, three approaches can be used individually or in combination to enhance bioavailability: (1) nanoparticle formation by Flash NanoPrecipitation, (2) co-administration with permeabilizers, and (3) enteric coating to minimize degradation in gastric fluids.

(1) Nanoparticle formation: Nanoparticles enhance bioavailability by two mechanisms. First, decreasing size increases the surface area per mass. For drugs like DHM that are dissolution limited, this increases the dissolution rate. Second, rapid precipitation processes, such as Flash NanoPrecipitation (FNP), can solidify the drug in an amorphous state rather than a crystalline state. The amorphous state has higher solubility than the crystalline state (Savjani, K. T.; Gajjar, A. K.; Savjani, J. K., “Drug solubility: importance and enhancement techniques”, ISRN Pharmaceutics 2012, 2012).

The nanoparticles can be produced by the polymer-directed or surfactant-directed rapid precipitation technique, Flash NanoPrecipitation, in embodiments that are described. To form DHM nanoparticles, the DHM is dissolved in a solvent phase (e.g., a water-miscible organic solvent) and rapidly mixed against an aqueous phase to effect particle nucleation and growth. The aqueous phase can include water as the solvent; however, the aqueous phase can instead or also include another polar solvent, such as methanol or ethanol or an alcohol/water mixture. That is, a solvent other than or in addition to water can be used as the aqueous phase, such an aqueous phase being more polar than the organic solvent phase. The stabilizer, which can be incorporated into the external aqueous phase or the solvent phase, arrests growth.

Solvents which can be used have been presented in U.S. Pat. No. 8,137,699. For example, the solvent (e.g., an organic solvent, an organic stream) can be completely miscible in the antisolvent stream (e.g., an aqueous solvent, an aqueous stream) under the conditions of the nanoparticle formation. (In some embodiments, the solvent could be partially soluble in the antisolvent stream.) For example, the ratio of the volume of the organic solvent to the ratio of the volume of the aqueous solvent that is mixed can be from about 0.01:1, 0.02:1, 0.05:1, 0.1:1, 0.2:1, 0.3:1, 0.5:1, 0.7:1, 0.8:1, 0.9:1, 1:1, 1:1.1, 1:1.2, 1:1.3, 1:1.5, 1:2, 1:3, 1:5, 1:10, 1:20, or 1:50 to about 0.02:1, 0.05:1, 0.1:1, 0.2:1, 0.3:1, 0.5:1, 0.7:1, 0.8:1, 0.9:1, 1:1, 1:1.1, 1:1.2, 1:1.3, 1:1.5, 1:2, 1:3, 1:5, 1:10, 1:20, 1:50, or 1:100. For example, the concentration of DHM in the organic stream can be from about 0.1, 0.2, 0.3, 0.5, 0.8, 1, 2, 3, 4, 5, 6, 8, 10, 12, 15, or 20 mg/mL to about 0.2, 0.3, 0.5, 0.8, 1, 2, 3, 4, 5, 6, 8, 10, 12, 15, 20, or 30 mg/mL. For example, the concentration of stabilizer in the organic stream or the aqueous stream can be from about 1, 2, 5, 8, 10, 12, 15, or 20 mg/mL to about 2, 5, 8, 10, 12, 15, 20, or 30 mg/mL. For example, the concentration of metal salt in the aqueous stream can be from about 0.1, 0.2, 0.5, 0.8, 1, 1.5, 2, 2.5, 3, or 5 mg/mL to about 0.2, 0.5, 0.8, 1, 1.5, 2, 2.5, 3, 5, or 10 mg/mL.

Following the mixing of the organic stream and the aqueous stream, the mixed stream from a CIJ or MIVM mixer can be collected in a reservoir containing liquid, which can optionally contain a buffer, such as PBS. For example, the ratio of the volume of the organic solvent plus the aqueous solvent to the volume of the reservoir liquid can be from about 1:1, 1:2, 1:3, 1:4, 1:5, 1:6, 1:8, 1:10, or 1:15 to about 1:2, 1:3, 1:4, 1:5, 1:6, 1:8, 1:10, 1:15, or 1:20. For example, the pH in the reservoir following receipt of the organic solvent and the aqueous solvent can be from about 4, 5, 5.5, 5.8, 6, 6.3, 6.4, 6.5, 6.7, 6.9, 7, 7.5. 8, 8.5, or 9 to about 5, 5.5, 5.8, 6, 6.3, 6.4, 6.5, 6.7, 6.9, 7, 7.5. 8, 8.5, 9, or 10.

Stabilizers include those amphiphilic block copolymers listed in U.S. Pat. No. 8,137,699, but also zein proteins as described by Weissmueller and Prud'homme (Weissmueller, N. T.; Lu, H. D.; Hurley, A.; Prud'homme, R. K., “Nanocarriers from GRAS zein proteins to encapsulate hydrophobic actives”, Biomacromolecules 2016, 17 (11), 3828-3837), hydroxypropyl methylcellulose (HPMC) in the class of succinic anyhydride modified cellulosics, and lecithin. Hydroxypropyl methylcellulose—acetate succinate (HPMCAS) polymers of the compositions according to the invention can have hydroxypropyl substitution levels of 5-10% wt; methoxyl substitution levels of 20-26% wt; acetyl substitutions of 5-14% wt (for example, 10-14% wt substitution); and succinyl substitutions of 4-18% wt (for example, 4-8% wt). Surfactants may also be used to stabilize the nanoparticles. Particularly effective surfactants are of the class of ethoxylated sugar-based surfactants, such as those sold by the trade names Tween® or Span®. Likewise, polymeric surfactants such as polyethylene oxide-block-polypropylene oxide (PEO-b-PPO) are also useful. Also, tocopherol substituted 2K PEG surfactants, usually denoted TPGS, may be used. The stabilizer may be added to either the organic or to the aqueous phases. Gelatin can be used as a stabilizer.

Cyclodextrins can be included in the nanoparticles according to the invention. For example, a cyclodextrin can include 5 or more glucose (e.g., α-D-glucopyranoside) units linked in a cycle, such as a (alpha)-cyclodextrin (6 units), β (beta)-cyclodextrin (7 units), and γ (gamma)-cyclodextrin (8 units), which are generally recognized as safe by the U.S. Food and Drug Administration (FDA). Cyclodextrins can form a toroidal or conical structure, and can have less hydrophilic or hydrophobic functionality in their interior and hydrophilic functionality on their exterior; thus, cyclodextrins can have surface-active properties. Cyclodextrins can act to host, sequester, or form complexes with hydrophobic compounds and can act to enhance drug permeability through mucosal tissue, i.e., cyclodextrins may act as permeabilizers.

The DHM itself is not hydrophobic enough to prepare in nanoparticle form, but it is known that the hydroxyls on tannic acid can be complexed with metal ions, which include, but are not limited to Fe (iron) cations, to produce nanoparticles (Tang, C.; Amin, D.; Messersmith, P. B.; Anthony, J. E.; Prud'homme, R. K., “Polymer Directed Self-Assembly of pH-Responsive Antioxidant Nanoparticles”, Langmuir 2015, 31 (12), 3612-3620). These same interactions occur for the tri-hydroxy ring on DHM. Using this iron coordination it is possible to make DHM nanoparticles. The coordination also ensures that the DHM is not in a crystalline state in the nanoparticle core. This ensures high supersaturation upon dissolution, which leads to high bioavailability. Molar ratios of DHM to iron cation can be in the range of from 0.01:1 to 10:1, for example, in the range of from 0.5:1 to 5:1. The mass ratio of DHM to stabilizer can be in the range of from 0.1:1 to 500:1, for example, in the range of from 1:1 to 50:1.

In an embodiment of the invention, a nanoparticle can include DHM in an amorphous state. For example, all DHM in the nanoparticle can be in an amorphous state. The entire nanoparticle can be in an amorphous state.

### EXAMPLES

Embodiments of the invention can include nanoparticles comprising

(1) dihydromyricetin stabilized by iron(III) complexation, (2) one or more permeabilizers, and/or (3) an enteric coating.

To form the DHM encapsulated nanoparticles, DHM can be dissolved in an organic solvent (the resultant organic solution can then be used as an organic stream). The candidates of organic solvent can be those described in U.S. Pat. No. 8,137,699. Embodiments include methanol, ethanol, n-propanol, isopropanol, acetone, ethyl acetate, tetrahydrofuran (THF), or mixtures of these as the organic solvent.

FNP can be performed using a mixing device such as a Confined Impinging Jet (CIJ) or a Multi-Inlet Vortex Mixer (MIVM). The CIJ used in the experiments consists of two opposed 0.5 mm jets of fluid, one organic and one aqueous, fed to a 2.4 mm diameter chamber at a constant rate with their momentum matched (D'addio, S. M et al. 2011; Saad, W. S. et al. 2016; U.S. Pat. No. 8,137,699). MIVM consists of four streams and allows control of both the supersaturation and the final solvent quality by varying stream velocities (Liu, Y.; Cheng, C. Y.; Prud'homme, R. K.; Fox, R. O., “Mixing in a multi-inlet vortex mixer (MIVM) for flash nano-precipitation”, Chemical Engineering Science 2008, 63 (11), 2829-2842). It is able to separate the reactive components into different streams prior to mixing.

The FNP process is facilitated when the hydrophobic materials encapsulated into the core of nanoparticles have a log P of more than 3.5. Log P of DHM is only approximately 1.31 (data collected by United States Environmental Protection Agency (EPA)). Therefore, it can be difficult to form nanoparticles by direct precipitation. Hence, a novel technique can be used to increase the hydrophobicity of DHM by complexing with iron cations. Such a technique can complex polyphenolic compounds with metal ions, which include, but are not limited to Fe(III) (Ji, Y. et al. 2001). The hydrophobic complex is able to be encapsulated into the core of nanoparticles. The nanoparticles produced by the FNP process in the following examples all have narrow distribution of sizes with polydispersity index generally less than 0.2.

Example 1: Iron(III) chloride was dissolved into an aqueous stream and rapidly mixed with an organic stream containing DHM and the amphiphilic stabilizer, so as to form nanoparticles. The particle formation was conducted with a CIJ mixer using 10 mL syringes to introduce the solvents. The first, organic stream contained 1 mg/mL of DHM and 10 mg/mL of PS1.6k-b-PEG5k stabilizer in THF. The second, aqueous stream contained 2 mg/mL of iron(III) chloride. Nanoparticles were collected in a reservoir with PBS buffer, so that the ratio of PBS buffer to the total output of the CIJ mixer was 4:1. The PBS buffer provided a final pH in the reservoir of pH=6.43. The resulting nanoparticles were 220 nm. Nanoparticles sizes changed less than 10 nm over the duration of 1 month.

Example 2: Using the same CIJ mixer, nanoparticles were formed from 2 mg/mL of DHM and 10 mg/mL of PS1.6k-b-PEG5k stabilizer in the THF stream and 2 mg/mL of iron(III) chloride in the aqueous stream. Nanoparticles sizes were 140 nm after dilution in PBS solution. The final pH was 6.27. Nanoparticles sizes changed less than 10 nm over 1 month.

Each iron(III) ion binds to three di-phenol groups at pH values higher than 8 (Fang, H.-L. 2007). In one example formulation, 2 mg/mL iron(III) chloride was added to the aqueous stream of FNP, producing pH of 2.3. Different methods may be used to adjust pH values during and after FNP to ensure hydrophobic complex formation and nanoparticle stability for at least a month in a 9:1 water to THF reservoir. Methods adopted to increase pH during the mixing of DHM and iron(III) include the addition of organic bases into the organic stream and addition of hydroxide into a separate aqueous stream in the operation of MIVM. The choice of organic bases includes, but is not limited to pyridine. If a MIVM mixer is used, then one aqueous stream can contain the Fe(III) at low pH to prevent oxide formation, and another aqueous stream can contain a high pH buffer or hydroxide concentration to create a mixed solution with a pH greater than 6, which will drive the Fe-DHM complexation. In one such example, the MIVM was operated to include one PBS stream, one water stream with iron(III) chloride dissolved, one potassium hydroxide water stream, and one THF stream including DHM and stabilizer. Methods adopted to increase pH after the mixing of DHM and iron(III) include the addition of PBS or ammonia base into the nanoparticle reservoir after FNP.

Example 3: Nanoparticles were made in the CIJ mixer. The organic stream consisted of 88 mg/mL of pyridine, 1 mg/mL of DHM, and 10 mg/mL of PS1.6k-b-PEG5k stabilizer in THF. The aqueous stream contained 2 mg/mL of iron(III) chloride. Nanoparticles were diluted 5× (five times) times in PBS solution, giving a pH=6.87. The nanoparticles produced were 190 nm.

Depending on the concentration of DHM in the organic stream and concentration of iron(III) in the water stream, nanoparticles of sizes from 20 to 300 nm are produced. The size of the nanoparticles increases with increasing concentration of iron(III) in the aqueous stream, but decreases with increasing concentration of DHM in the organic stream. Therefore, by varying the concentration of both, nanoparticles of a required size can be produced, which can be used under different requirements of DHM delivery.

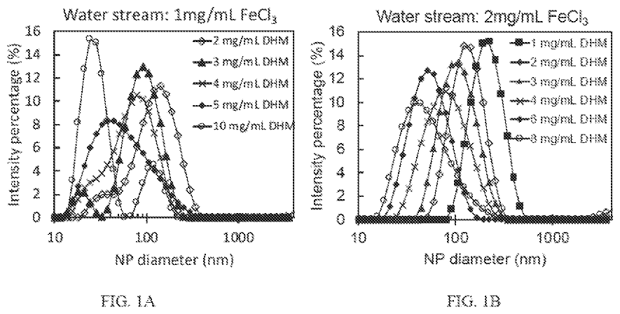

Example 4: Nanoparticle size control. Nanoparticles were made in the CIJ mixer with the aqueous phase held constant at 1 mg/mL FeCl₃, while the DHM concentration in the THF solvent stream was varied between 2-10 mg/mL of DHM. The concentration of PS1.6k-b-PEG5k stabilizer in the THF stream was kept at 10 mg/mL. Nanoparticles were collected in a reservoir with PBS buffer such that the ratio of PBS buffer to the total output of the CIJ mixer was 4:1. The results are shown in FIG. 1. Particle sizes could be controlled between 50 and 150 nm.

Example 5: Nanoparticle size control. Nanoparticles were made in the CIJ mixer with the aqueous phase held constant at 2 mg/mL FeCl₃, while the DHM concentration in the THF solvent stream was varied between 1-8 mg/mL of DHM. The concentration of PS1.6k-b-PEG5k stabilizer in the THF stream was kept at 10 mg/mL. Nanoparticles were collected in a reservoir with PBS buffer, so that the ratio of PBS buffer to the total output of the CIJ mixer was 4:1. The size distribution results are shown in FIGS. 1A and 1B. Peak particle sizes were controlled between 50 and 220 nm. FIGS. 1A and 1B show particle size distributions obtained for different DHM to iron(III) chloride ratios. Comparison of FIG. 1A, which shows results obtained with a concentration of iron(III) chloride (FeCl₃) in the aqueous (water) stream of 1 mg/mL, with FIG. 1B, which shows results obtained with a concentration of iron(III) chloride (FeCl₃) in the aqueous (water) stream of 2 mg/mL, shows that for a fixed concentration of DHM in the THF stream, nanoparticle sizes increase with increasing concentration of iron(III) chloride in the aqueous stream. For a fixed concentration of iron(III) chloride in the aqueous stream, nanoparticle sizes increase with decreasing concentration of DHM in the THF stream.

Example 6: Acetone was used as the solvent for the organic stream. Nanoparticles were made in the CIJ mixer with the aqueous phase held constant at 2 mg/mL FeCl₃, while the DHM concentration in the acetone solvent stream was 2 mg/mL. Nanoparticles were collected in a reservoir with PBS buffer, so that the ratio of PBS buffer to the total output of the CIJ mixer was 4:1. The concentration of PS1.6k-b-PEG5k stabilizer in the THF stream was kept at 10 mg/mL. The resultant particle size was 110 nm.

Example 7: Bio-degradable hydroxypropyl methylcellulose (HPMC) was used as a stabilizer in the organic stream. Nanoparticles were made in the CIJ mixer with the aqueous phase held constant at 2 mg/mL FeCl₃, while the DHM concentration in the THF or acetone solvent stream was 1-2 mg/mL. In the THF or acetone organic stream, there were 10 mg/mL hydroxypropyl methylcellulose acetate succinate (HPMCAS) 126. Nanoparticles were collected in a reservoir with PBS buffer, so that the ratio of PBS buffer to the total output of the CIJ mixer was 4:1. Using THF as the organic stream, the size of the nanoparticles was 320 nm for 1 mg/mL DHM in the THF stream and 290 nm for 2 mg/mL DHM in the THF stream. Using acetone as the organic stream, the size of the nanoparticles was 180 nm for 2 mg/mL DHM in the acetone stream. Thus, switching from PS1.6k-b-PEG5k to HPMCAS126 as the stabilizer, while keeping the rest of the nanoparticle formulation the same, resulted in an increase in the size of the nanoparticles.

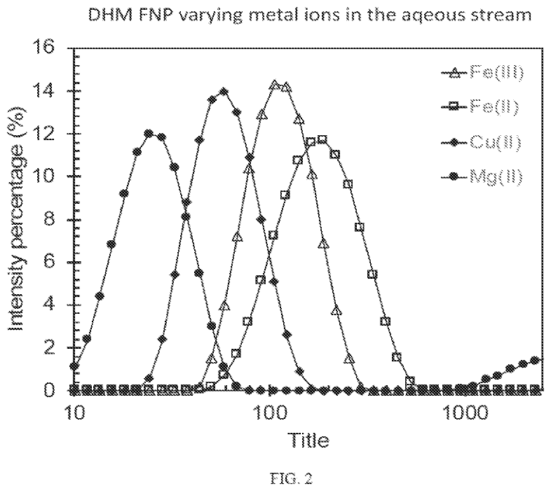

Example 8: 2 mg/mL of Fe(II)Cl₂, Cu(II)Cl₂, or Mg(II)Cl₂ was added into the aqueous stream. 2 mg/mL of DHM and 10 mg/mL PS1.6k-b-PEG5k was added into the acetone stream. The stream coming out of the CIJ was collected in a PBS reservoir with 4 times volume. Use of Fe(II)Cl₂ produced nanoparticles of 150 nm size; use of Cu(II)Cl₂ produced nanoparticles of 60 nm size; and use of Mg(II)Cl₂ produced nanoparticles of 25 nm size. Use of Fe(III)Cl₂ produced nanoparticles of 110 nm size. FIG. 2 shows the size and size distribution of nanoparticles made through FNP with each of these metal ions in the aqueous stream. Table 1 gives the z-average number and PDI for each formulation variation.

TABLE 1
Size of and PDI for DHM nanoparticles made using various metal ions,
stabilizers, and organic streams (solvents). The metal ion concentration
in the aqueous stream is 2 mg/mL, and the concentration of stabilizers
in the respective organic stream is 10 mg/mL.

| | | Organic | Nanoparticle | |
|---|---|---|---|---|
| Metal ion | Stabilizer | solvent | size (nm) | PDI |
| Fe(III) | PS1.6k-b-PEG5k | acetone | 110 | 0.11 |
| Fe(II) | PS1.6k-b-PEG5k | acetone | 150 | 0.20 |
| Cu(II) | PS1.6k-b-PEG5k | acetone | 60 | 0.21 |
| Mg(II) | PS1.6k-b-PEG5k | acetone | 25 | 0.33 |
| Fe(III) | HPMCAS126 | acetone | 180 | 0.13 |
| Fe(II) | HPMCAS126 | acetone | 170 | 0.12 |
| Cu(II) | HPMCAS126 | acetone | 400 | 0.31 |
| Fe(III) | PS1.6k-b-PEG5k | THF | 120 | 0.16 |
| Fe(II) | PS1.6k-b-PEG5k | THF | 100 | 0.14 |
| Cu(II) | PS1.6k-b-PEG5k | THF | 60 | 0.17 |
| Fe(III) | HPMCAS126 | THF | 210 | 0.18 |
| Fe(II) | HPMCAS126 | THF | 200 | 0.17 |
| Cu(II) | HPMCAS126 | THF | 300 | 0.27 |

The encapsulation efficiency of DHM into nanoparticles produced using FNP is defined as the mass of DHM encapsulated into the nanoparticles over the total mass added to the organic stream prior to FNP. For example, the encapsulation efficiency of DHM into nanoparticles according to the present invention can be from about 10%, 20%, 30%, 40%, 50%, 60%, 63%, 65%, 70%, 72%, 75%, 80%, 85%, 90%, 94%, 95%, 96%, 98%, 99%, 99.5%, or 99.8% to about 20%, 30%, 40%, 50%, 60%, 63%, 65%, 70%, 72%, 75%, 80%, 85%, 90%, 94%, 95%, 96%, 98%, 99%, 99.5%, 99.8%, or 100%.

DHM can exist in three states in a nanoparticle solution, namely free DHM, a DHM-metal complex (e.g., a DHM-iron(III) complex) directly dissolved in solvent, or encapsulated DHM in nanoparticles. DHM in the latter state can be separated from the previous two states using an Amicon® 100k centrifugal ultrafilter. Unencapsulated DHM and DHM-iron(III) complex come through the filter into the supernatant solution. The absorbance spectrum of this supernatant solution is determined using a SpectraMax® i3x plate reader.

For example, there are three different ways to calibrate the concentration of DHM based on its absorbance spectrum: (1) calibrate at the natural pH condition (0.5 mg/mL DHM gives pH=5.8); (2) calibrate at pH=2; or (3) calibrate at pH=11.

For calibration method (1): Calibration curves of absorbance for different concentrations of DHM in a solvent mixture of 9:1 water to THF were made. The peak was seen at 325 nm. A calibration curve of iron(III) chloride together with DHM in a solvent mixture of 9:1 water to THF was made. The only peak in this case was at 295 nm, up to a mass ratio of DHM to iron(III) chloride reaching 10, where another peak at 325 nm appears. That means that when the mass ratio of iron(III) chloride to DHM is larger than 10, the complex formation between DHM and iron(III) has reached saturation, so that free DHM starts to appear. For the same concentration of DHM, given that the complex formation has not yet reached saturation, the variation of iron(III) concentration does not affect the absorbance at 295 nm or 325 nm. Hence, the linear relation of absorbance over concentration of DHM for both free DHM and DHM in the form of DHM-iron(III) complex at both 295 nm and 325 nm (four linear correlations in total) was determined. Let C be the concentration of DHM in the THF stream in mg/mL. For the absorbance of free DHM at 295 nm, absorbance=22.71*C+0.06; for the absorbance of free DHM at 325 nm, absorbance=57.82*C−0.02. For the absorbance of DHM-iron(III) complex at 295 nm, absorbance=42.20*C−0.07; for the absorbance of DHM-iron (III) complex at 325 nm, absorbance=13.80*C+0.08.

Given the absorbance spectrum of the supernatant, it is possible to calculate the concentration of dissolved DHM and dissolved DHM-iron(III). The overall encapsulation efficiency can then be calculated. The encapsulation efficiency is defined as the amount of DHM encapsulated within the nanoparticles over the total amount of DHM added to the organic stream before FNP.

Example 9: Encapsulation efficiency. A formulation consisted of 1 mg/mL of DHM and 10 mg/mL of PS1.6k-b-PEG5k dissolved in 1 mL THF stream and 2 mg/mL of iron(III) chloride dissolved in 1 mL deionized (DI) water. These were rapidly mixed in a CIJ mixer and further diluted in an additional 8 mL PBS reservoir.

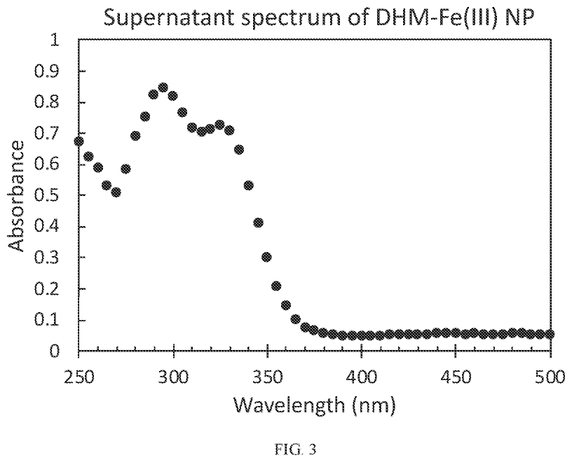

The encapsulation efficiency for DHM nanoparticles made of different formulations was compared. The formulation with 1 mg/mL of DHM and 10 mg/mL of PS1.6k-b-PEG5k in the THF stream and 2 mg/mL of Fe(III) chloride in the aqueous stream yielded an encapsulation efficiency of 76%, as shown in FIG. 3. By adding 88 mg/mL of pyridine into the organic stream, the pH of the final solution was increased, resulting in improved hydrophobic complex formation and a higher encapsulation efficiency of 88%. By adding 1 mg/mL more of DHM into the THF stream, the unencapsulated DHM increased to 0.0352 mg/mL. The encapsulation efficiency increased from 76% to 82.5%, because of the higher amount of initial DHM input (0.2 mg/mL in the PBS reservoir).

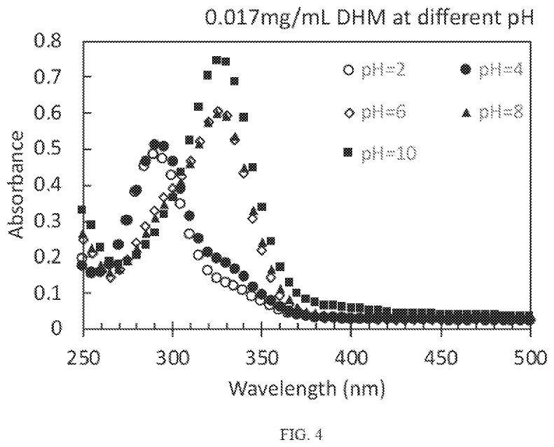

For calibration methods (2) and (3): Experiments were conducted that indicated the shift of the DHM absorbance peak from 290 nm to 325 nm as the pH increased, as shown in FIG. 4. Water with the pH adjusted by an acid, such as hydrochloric acid (HCl) and/or trifluoroacetic acid (TFA), was added to adjust the pH of DHM solution to 2 (method 2), or water with the pH adjusted by a base, such as sodium hydroxide (NaOH) and/or potassium hydroxide (KOH), was added to the DHM solution to increase its pH to 10. The absorbance of DHM=33*concentration of DHM (mg/mL)+0.02 for pH <2 medium at the peak of 290 nm, and the absorbance of DHM=52*concentration of DHM (mg/mL)+0.02 for pH >10 medium at the peak of 325 nm. The choices of solvent for DHM include, but are not limited to water, a water and THF mixture, and ethyl acetate. The correlation of absorbance to concentration differs by less than 5%. The addition of Fe(II), Fe(III), and Cu(II) does not affect the absorbance peak value of DHM at 290 nm.

Example 10: A formulation consisted of 2 mg/mL of DHM and 10 mg/mL of PS1.6k-b-PEG5k or HPMCAS126 dissolved in a 1 mL acetone stream and 2 mg/mL of Fe(III) chloride, Fe(II) chloride, or Cu(II) chloride dissolved in 1 mL DI water. These were rapidly mixed in a CU mixer and further diluted in an additional 8 mL PBS reservoir. The supernatant of nanoparticle solution was filtered and dried to get rid of acetone, whose absorbance peak overlaps with that of DHM. The dried supernatant was re-dispersed using 9:1 water:THF and mixed with dilute HCl to achieve a final pH of 2, and the concentration of unencapsulated DHM was back calculated based on the correlation between the absorbance of DHM and DHM concentration at pH around 2. The calculated encapsulation efficiency is listed in Table 2. Nanoparticles made using Fe(II) and Cu(II) in the aqueous stream during FNP had higher encapsulation efficiencies.

TABLE 2
Comparison of the encapsulation efficiency of
different DHM-metal nanoparticle formulations.

| | | Organic | Encapsulation |
|---|---|---|---|
| Metal ion | Stabilizer | solvent | efficiency |
| Fe(III) | PS1.6k-b-PEG5k | acetone | 72% |
| Fe(II) | PS1.6k-b-PEG5k | acetone | 94% |
| Cu(II) | PS1.6k-b-PEG5k | acetone | 96% |
| Fe(III) | HPMCAS126 | acetone | 63% |
| Fe(II) | HPMCAS126 | acetone | 96% |
| Cu(II) | HPMCAS126 | acetone | 95% |

Specific techniques have been developed to dry the nanoparticle solution made through FNP. Dried forms of DHM-metal nanoparticles (NPs) can be redispersed in water without a significant change of the nanoparticle size distribution.

Example 12: A formulation consisted of 2 mg/mL of DHM and 10 mg/mL of PS1.6k-b-PEG5k dissolved in 1 mL acetone stream and 2 mg/mL of Fe(III) chloride or Fe(II) chloride dissolved in 1 mL DI water. These were rapidly mixed in a CIJ mixer and further diluted in an additional 8 mL PBS reservoir. The nanoparticles were first dialysised overnight to eliminate acetone solvent. A 1:1 mass ratio of cyclodextrin:nanoparticle was added before lyophilization. After re-dispersion of the lyophilized nanoparticle (NP) solid in water, it was observed that the size of Fe(III)-DHM nanoparticles increased from 110 to 150 nm, and the size of Fe(II)-DHM nanoparticles increased from 150 to 190 nm. In both cases, the PDI had a low value of 0.2, indicating narrow distributions of nanoparticle sizes.

(2) Permeation enhancers (permeabilizers): Permeation enhancers are agents that increase the transport of drugs across epithelial layers in the GI (gastrointestinal) tract. They have been reviewed by Aungst and Whitehead (Aungst, B. J., “Absorption enhancers: applications and advances”, The AAPS Journal 2012, 14 (1), 10-18; Thanou, M.; Verhoef, J.; Junginger, H., “Oral drug absorption enhancement by chitosan and its derivatives”, Advanced Drug Delivery Reviews 2001, 52 (2), 117-126; Whitehead, K.; Karr, N.; Mitragotri, S., “Safe and effective permeation enhancers for oral drug delivery”, Pharmaceutical Research 2008, 25 (8), 1782-1788; Whitehead, K.; Mitragotri, S., “Mechanistic analysis of chemical permeation enhancers for oral drug delivery”, Pharmaceutical Research 2008^{b}, 25 (6), 1412-1419). The list of agents presented by Aungst in Table I and Whitehead in Table I are incorporated into this patent in their entirety. Among these, a permeabilizer is capric acid and salts thereof. It is currently clinically approved for use in an ampicillin suppository. The caprates and other long-chain saturated acids and their salts can be directly incorporated into the nanoparticle during Flash NanoPrecipitation. Their hydrophobicity can be enhanced by complexing them with divalent cations such as magnesium, calcium, zinc, and divalent iron, or trivalent iron. Permeabilizers are optional additions to the formulation. When they are used, the mass ratios of permeabilizer to DHM can range from 1:100 to 100:1.

The final formulation may have mass ratios of permeabilizer to total mass of permeabilizer plus DHM of 0-1%. The final formulation may have mass ratios of permeabilizer to total mass of permeabilizer plus DHM of 0-2%. The final formulation may have mass ratios of permeabilizer to total mass of permeabilizer plus DHM of 0-10%. The final formulation may have mass ratios of permeabilizer to total mass of permeabilizer plus DHM of 0-5 0%. The final formulation may have mass ratios of permeabilizer to total mass of permeabilizer plus DHM of 0-75%. The final formulation may have mass ratios of permeabilizer to total mass of permeabilizer plus DHM of 0-90%. The final formulation may have mass ratios of permeabilizer to total mass of permeabilizer plus DHM of 0-99%.

The permeation enhancers can be incorporated into the DHM nanoparticle, or they can be included in the formulation in combination with the nanoparticles.

(3) Enteric coatings: It is reported that DHM is degraded by exposure to the low pH of gastric fluids. It is, therefore, desirable to protect the DHM from dissolution in the stomach. This can be accomplished by encapsulation of the DHM with an enteric polymer. Enteric polymers based on methacrylate copolymers can be used. A series of these polymers are made by Evonik Inc., Dusseldorf, Germany under the trade names Eudragit: E100; 5100; E PO; NE 40D; RI, PO; E PO Readymix; NM 30D; Plasacryl T20; L 100; RS PO; L100-55; Plasacryl HTP20; FS 30D. The Eudragit polymers with anionic groups can be used to complex with the Fe (iron) ions used to encapsulate DHM. This makes a single phase, enteric nanoparticle. Alternatively, the Eudragit polymers can be precipitated on preformed DHM nanoparticles by performing a second Flash NanoPrecipitation (FNP) process, where the Eudragit is introduced in a fluid stream and is precipitated by an antisolvent stream that contains the nanoparticles. The solvent stream into which the Eudragit is dissolved can be an aqueous soluble organic solution, or it may be an aqueous stream under pH conditions where the Eurdragit is soluble, and the antisolvent stream can be an aqueous stream with buffer capacity to change the final aqueous stream to a pH where the Eudragit precipitates.

Alternatively, the Eudragit coating can be applied by spray drying or spray coating as is practiced in the enteric coating of tablets.

Thus, a nanoparticle including DHM according to the invention can have no enteric coating; or the nanoparticle including DHM can have an enteric coating. Multiple (for example, many) nanoparticles can be grouped into an oral dosage form. That oral dosage form can have no enteric coating; or that oral dosage form can have an enteric coating.

The enteric coating is an optional component.

For example, the mass ratio of enteric polymer to DHM can be 0-5%. Alternatively, the mass ratio of enteric polymer to DHM can be 0-25%. Alternatively the mass ratio of enteric polymer to DHM can be 0-50%. Alternatively, the mass ratio of enteric polymer to DHM can be 0-90%.

The formulation of nanoparticles may also involve drugs in addition to DHM in its core, so as to achieve a better combined treatment for hangover in its application. Such drug candidates include and are not limited to Silymarin.

The nanoparticles and permeabilizers may be incorporated with other excipients that aid in granulation processes. The liquid nanoparticle dispersion may be processed into dry powder form by precipitation or drying processes. For example, a process to produce dry powders is spray drying. In the spray drying process excipients may be added to enhance the redispersion and bioavailability of the active agents. Excipients may include sugars such as trehalose, maltodextrin, sucrose, mannitol, or leucine, or casein, starches or cellulosic polymers.

For example, the nanoparticle formulation of DHM can increase bioavailability, as measured by mouse serum assay, by at least 100%. The nanoparticle formulation of DHM can increase bioavailability, as measured by mouse serum assay, by 50%. The nanoparticle formulation of DHM can increase bioavailability, as measured by mouse serum assay, by 15% (Tong, Q.; Hou, X.; Fang, J.; Wang, W.; Xiong, W.; Liu, X.; Xie, X.; Shi, C., “Determination of dihydromyricetin in rat plasma by LC-MS/MS and its application to a pharmacokinetic study”, Journal of Pharmaceutical and Biomedical Analysis 2015, 114, 455-461).

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A nanoparticle comprising dihydromyricetin complexed with a metal.

2. The nanoparticle of claim 1, wherein the dihydromyricetin is in an amorphous state.

3. The nanoparticle of claim 1, wherein the metal is iron (Fe) or iron(III) (Fe(III)).

4. (canceled)

5. The nanoparticle of claim 1, wherein the metal is iron(II) (Fe(II)).

6. The nanoparticle of claim 1, wherein the metal is copper (Cu) or copper(II) (Cu(II)).

7. (canceled)

8. The nanoparticle of claim 1, wherein the metal is magnesium (Mg).

9. The nanoparticle of claim 1, further comprising a stabilizer selected from the group consisting of an ethoxylated sugar surfactant, a block copolymer, polyethylene oxide-block-polypropylene oxide (PEO-b-PPO), a zein-casein protein mixture, gelatin, derivatized cellulosic polymer, hydroxypropyl methylcellulose with succinic anhydride substitution, polyethylene glycol (PEG) functionalized vitamin E (tocopherol succinate PEG), and combinations.

10. The nanoparticle of claim 1, further comprising polystyrene-block-polyethylene glycol (PS-b-PEG).

11. The nanoparticle of claim 1, further comprising a stabilizer selected from the group consisting of hydroxypropyl methylcellulose (HPMC), hydroxypropyl methylcellulose acetate succinate (HPMCAS), a cyclodextrin, and combinations.

12.-14. (canceled)

15. The nanoparticle of claim 1, encapsulated by an enteric coating, a polymeric coating, or a methacrylate copolymer coating.

16. The nanoparticle of claim 1, having a diameter in the range of from 10 nm to 1000 nm, in the range of from 20 nm to 500 nm, in the range of from 25 nm to 400 nm, in the range of from 60 nm to 400 nm, or in the range of from 100 to 250 nm.

17. An oral dosage form comprising the nanoparticle of claim 1.

18.-52. (canceled)

53. The method of claim 67, comprising treating a patient suffering from hangover symptoms, and comprising administering the nanoparticle of claim 1 to the patient to reduce the hangover symptoms.

54. The method of claim 67, comprising treating a patient at risk of an alcohol use disorder, and comprising administering the nanoparticle of claim 1 to the patient to prevent the alcohol use disorder.

55. The method of claim 67, comprising treating a patient at risk of alcoholism, and comprising administering the nanoparticle of claim 1 to the patient to prevent alcoholism.

56. The method of claim 67, comprising treating a patient suffering from an alcohol use disorder, and comprising administering the nanoparticle of claim 1 to the patient to treat the alcohol use disorder.

57. The method of claim 67, comprising treating a patient suffering from an alcoholism, and comprising administering the nanoparticle of claim 1 to the patient to treat the alcoholism.

58. The method of claim 67, comprising treating a patient suffering from an alcohol overdose, and comprising administering the nanoparticle of claim 1 to the patient to treat the alcohol overdose.

59. A method of treating a patient in need of increased antioxidant capacity, neuroprotection, protection of a kidney, protection of a liver, at risk of cancer, or suffering from inflammation, cancer, a metabolic disorder, or a bacterial infection comprising administering the nanoparticle of claim 1 to the patient to increase the antioxidant capacity, as neuroprotection, to protect the kidney, to protect the liver, to prevent the cancer, to inhibit the inflammation, to treat the cancer, to ameliorate the metabolic disorder, or to treat the bacterial infection.

60.-66. (canceled)

67. A method of treating a patient in need of alcohol-related healthcare, comprising administering the nanoparticle of claim 1 to the patient as the alcohol-related healthcare.
